(19) United States
(12) Patent Application Publication
(10) Pub. No.: US 2014/0142603 A1
(43) Pub. Date: May 22, 2014

(54) MICROSURGICAL INSTRUMENT HANDLE

(71) Applicant: Katalyst Surgical, LLC, Chesterfield, MO (US)

(72) Inventors: Gregg D. Scheller, Wildwood, MO (US); Matthew N. Zeid, Ballwin, MO (US)

(73) Assignee: Katalyst Surgical, LLC, Chesterfield, MO (US)

(21) Appl. No.: 14/078,148

(22) Filed: Nov. 12, 2013

Related U.S. Application Data
(60) Provisional application No. 61/727,901, filed on Nov. 19, 2012.

(51) Int. Cl. A61B 17/29; A61B 17/3201; A61B 17/00

(52) U.S. Cl. CPC A61B 17/2909; A61B 17/00234; A61B 17/3201
USPC 606/174; 606/1; 606/205

(57) ABSTRACT

A microsurgical instrument handle may include an actuation structure having an actuation structure distal end and an actuation structure proximal end, a plurality of actuation arms of the actuation structure, and a handle base having a handle base proximal end and a handle base distal end. Each actuation arm of the plurality of actuation arms may include an extension joint. A compression of the actuation structure may be configured to extend the actuation structure distal end relative to the actuation structure proximal end. A decompression the actuation structure may be configured to retract the actuation structure distal end relative to the actuation structure proximal end.

### CROSS-REFERENCE TO RELATED APPLICATIONS

This Application claims the benefit of U.S. Provisional Application No. 61/727,901, filed Nov. 19, 2012.

### FIELD OF THE INVENTION

The present disclosure relates to a medical device, and, more particularly, to a surgical instrument.

### BACKGROUND OF THE INVENTION

A variety of surgical procedures are performed through a very small surgical incision in a particular tissue. Reducing the size of a surgical incision during a surgical procedure generally reduces the amount of trauma to the surgical site and generally facilitates faster wound healing. In order to perform surgical procedures through a very small surgical incision, a surgeon may require specialized surgical instruments configured to fit through the very small surgical incision and provide the surgeon with a surgical utility. Sometimes a surgeon may require a surgical utility that may not be easily controlled close to a particular surgical site, e.g., closing forceps jaws inside of an eye. It is generally desirable for a surgeon to be able to control such a surgical utility with a minimal amount of effort. For example, if a surgical utility is controlled by a lever or a switch on an instrument handle, a surgeon may need to adjust an orientation of a surgical instrument in order to actuate the lever or the switch. Additionally, if a surgical utility control mechanism requires a surgeon to apply a significant amount of force to a portion of a surgical instrument, then it may be difficult for the surgeon to manipulate the surgical utility control mechanism without unintentionally moving a portion of the surgical instrument.

However, it is important that some effort is required to manipulate a surgical utility control mechanism of a surgical instrument. For example, if manipulation of a surgical utility control mechanism only requires a surgeon to apply a very small force to a portion of a surgical instrument, then it may be possible for the surgeon to unintentionally manipulate a surgical utility control mechanism during a surgical procedure. Accordingly, there is a need for a surgical instrument handle to control a surgical utility through a very small surgical incision with an optimal amount of effort.

### BRIEF SUMMARY OF THE INVENTION

The present disclosure presents a microsurgical instrument handle. In one or more embodiments, a microsurgical instrument handle may comprise an actuation structure having an actuation structure distal end and an actuation structure proximal end, a plurality of actuation arms of the actuation structure, and a handle base having a handle base proximal end and a handle base distal end. Illustratively, each actuation arm of the plurality of actuation arms may comprise an extension joint. In one or more embodiments, a compression of the actuation structure may be configured to extend the actuation structure distal end relative to the actuation structure proximal end. Illustratively, a decompression the actuation structure may be configured to retract the actuation structure distal end relative to the actuation structure proximal end.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A, 1B, 1C, and 1D are schematic diagrams illustrating an actuation structure;

FIG. 2 is a schematic diagram illustrating an exploded view of a surgical instrument assembly;

FIGS. 3A, 3B, and 3C are schematic diagrams illustrating a gradual closing of a surgical tool;

FIGS. 4A, 4B, and 4C are schematic diagrams illustrating a gradual opening of a surgical tool.

### DETAILED DESCRIPTION OF AN ILLUSTRATIVE EMBODIMENT

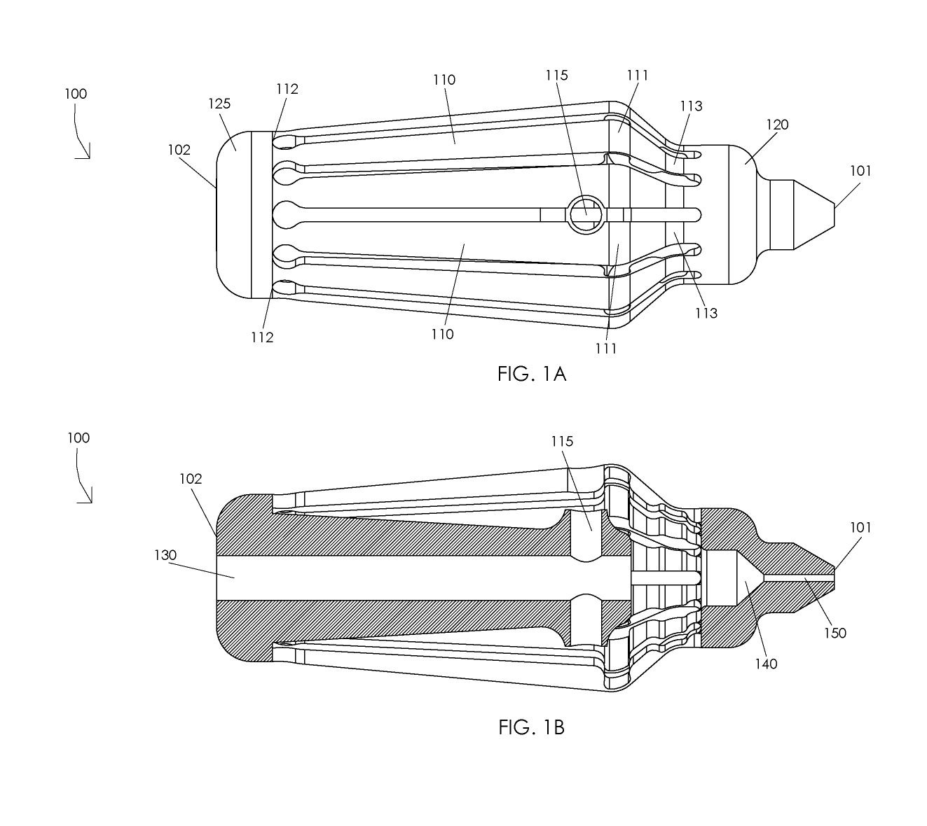

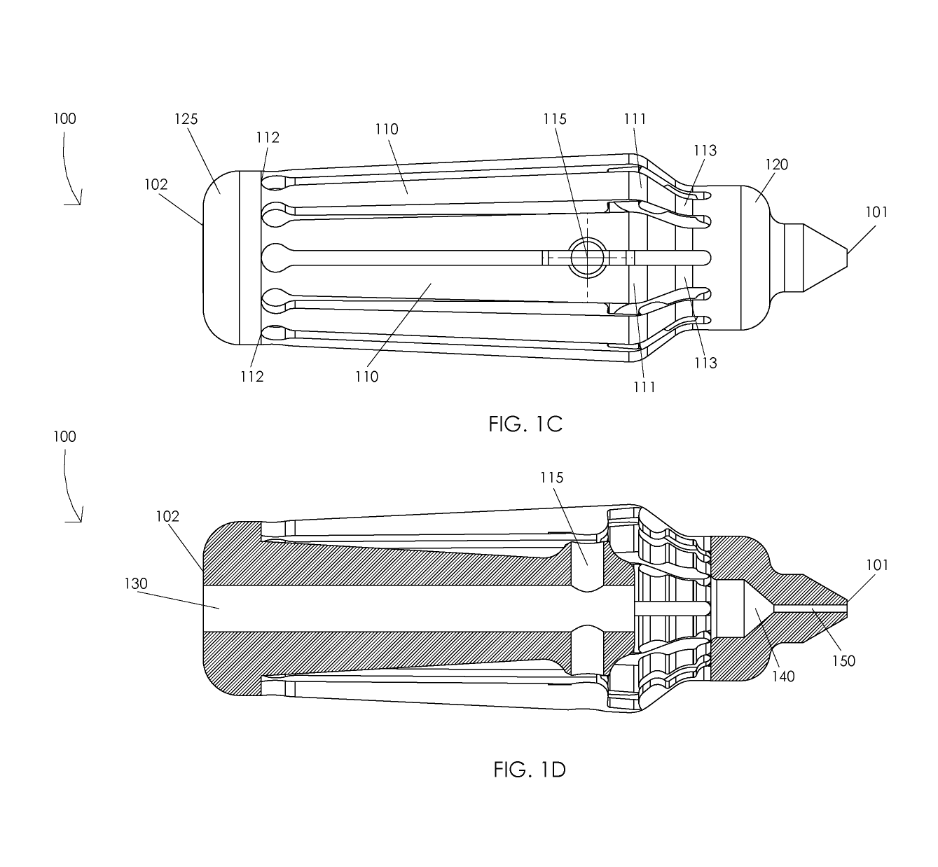

FIGS. 1A, 1B, 1C, and 1D are schematic diagrams illustrating an actuation structure 100. FIG. 1A illustrates a top view of a decompressed actuation structure 100. Illustratively, actuation structure 100 may comprise an actuation structure distal end 101 and an actuation structure proximal end 102, a plurality of actuation arms 110, a fixation mechanism housing 115, an actuation arm distal interface 120, and an actuation arm proximal interface 125. In one or more embodiments, each actuation arm 110 of a plurality of actuation arms 110 may comprise an extension joint 111, a proximal extension hinge 112, and a distal extension hinge 113. Illustratively, actuation structure distal end 101 may extend a decompressed distance from actuation structure proximal end 102, e.g., when actuation structure 100 comprises a decompressed actuation structure 100. In one or more embodiments, a decompressed distance may be in a range of 1.6 to 3.0 inches, e.g., a decompressed distance may be 2.25 inches. Illustratively, a decompressed distance may be less than 1.6 inches or greater than 3.0 inches.

FIG. 1B illustrates a cross-sectional view of a decompressed actuation structure 100. Illustratively, an actuation structure 100 may comprise an inner bore 130, an inner bore distal taper 140, and an actuation sleeve housing 150. In one or more embodiments, actuation structure 100 may be manufactured from any suitable material, e.g., polymers, metals, metal alloys, etc., or from any combination of suitable materials. Illustratively, actuation structure 100 may be manufactured from a shape memory material. In one or more embodiments, actuation structure 100 may be manufactured using a selective laser sintering machine. Illustratively, actuation structure 100 may be manufactured by additive manufacturing or 3D printing.

In one or more embodiments, actuation structure 100 may have a density in a range of 0.02 to 0.06 pounds per cubic inch, e.g., actuation structure 100 may have a density of 0.042 pounds per cubic inch. Illustratively, actuation structure 100 may have a density less than 0.02 pounds per cubic inch or greater than 0.06 pounds per cubic inch. In one or more embodiments, actuation structure 100 may have a mass in a range of 0.005 to 0.025 pounds, e.g., actuation structure 100 may have a mass of 0.015 pounds. Illustratively, actuation structure 100 may have a mass less than 0.005 pounds or greater than 0.025 pounds. In one or more embodiments, actuation structure 100 may have a volume in a range of 0.2 to 0.5 cubic inches, e.g., actuation structure 100 may have a volume of 0.359 cubic inches. Illustratively, actuation structure 100 may have a volume less than 0.2 cubic inches or greater than 0.5 cubic inches. In one or more embodiments, actuation structure 100 may have a surface area in a range of 7.5 to 13.0 square inches, e.g., actuation structure 100 may have a surface area of 10.8 square inches. Illustratively, actuation structure 100 may have a surface area less than 7.5 square inches or greater than 13.0 square inches.

In one or more embodiments, actuation structure 100 may be manufactured from a material suitable for sterilization by a medical autoclave. Illustratively, actuation structure 100 may be manufactured from a material, e.g., Nylon, configured to withstand exposure to temperatures, pressures, and ambient conditions present in a medical autoclave without degradation. For example, actuation structure 100 may be configured to function normally after exposure in a temperature 250° F. for 15 minutes at an atmospheric pressure of 15 psi. In one or more embodiments, actuation structure 100 may be configured to be used in a surgical procedure and then sterilized by a medical autoclave at least three times. Illustratively, actuation structure 100 may be configured to be used in a surgical procedure and then sterilized by a medical autoclave more than three times.

FIG. 1C illustrates a top view of a compressed actuation structure 100. FIG. 1D illustrates a cross-sectional view of a compressed actuation structure 100. In one or more embodiments, actuation structure 100 may be configured to project actuation structure distal end 101 a first distance from actuation structure proximal end 102, e.g., when actuation structure 100 is fully decompressed. Illustratively, actuation structure 100 may comprise a shape memory material configured to project actuation structure distal end 101 a second distance from actuation structure proximal end 102, e.g., when actuation structure 100 is fully compressed. In one or more embodiments, the second distance from actuation structure proximal end 102 may be greater than the first distance from actuation structure proximal end 102. Illustratively, a compression of actuation structure 100 may be configured to gradually extend actuation structure distal end 101 relative to actuation structure proximal end 102.

In one or more embodiments, actuation structure distal end 101 may extend a compressed distance from actuation structure proximal end 102, e.g., when actuation structure 100 comprises a compressed actuation structure 100. Illustratively, a compressed distance may be a distance in a range of 1.6 to 3.0 inches, e.g., a compressed distance may be 2.29 inches. In one or more embodiments, a compressed distance may be less than 1.6 inches or greater than 3.0 inches. Illustratively, a compressed distance may be in a range of 0.02 to 0.05 inches greater than a decompressed distance. In one or more embodiments, a compressed distance may be less than 0.02 inches greater than a decompressed distance. Illustratively, a compressed distance may be more than 0.05 inches greater than a decompressed distance. In one or more embodiments, a compressed distance may be in a range of 1.0 to 2.0 percent greater than a decompressed distance. Illustratively, a compressed distance may be less than 1.0 percent greater than a decompressed distance. In one or more embodiments, a compressed distance may be more than 2.0 percent greater than a decompressed distance.

Illustratively, actuation structure 100 may be compressed by an application of a force, e.g., a compressive force, to a portion of actuation structure 100. In one or more embodiments, an application of a compressive force in a range of 0.2 to 1.0 pounds may be configured to compress actuation structure 100, e.g., an application of a compressive force of 0.84 pounds may be configured to compress actuation structure 100. Illustratively, an application of a compressive force of less than 0.2 pounds or greater than 1.0 pounds may be configured to compress actuation structure 100. In one or more embodiments, actuation structure 100 may be compressed by an application of one or more compressive forces at one or more locations around an outer perimeter of actuation structure 100. Illustratively, the one or more locations may comprise any particular locations of a plurality of locations around an outer perimeter of actuation structure 100. For example, a surgeon may compress actuation structure 100 by squeezing actuation structure 100. Illustratively, a surgeon may compress actuation structure 100 by squeezing actuation structure 100 at any particular location of a plurality of locations around an outer perimeter of actuation structure 100.

In one or more embodiments, a surgeon may compress actuation structure 100 by applying a force to a portion of actuation structure 100, e.g., when actuation structure 100 is in a first rotational orientation. Illustratively, the surgeon may then rotate actuation structure 100 and compress actuation structure 100 by applying a force to a portion of actuation structure 100, e.g., when actuation structure 100 is in a second rotational orientation. In one or more embodiments, the surgeon may then rotate actuation structure 100 and compress actuation structure 100 by applying a force to a portion of actuation structure 100, e.g., when actuation structure 100 is in a third rotational orientation. Illustratively, a surgeon may compress actuation structure 100 by applying a force to a portion of actuation structure 100, e.g., when actuation structure 100 is in any rotational orientation.

In one or more embodiments, actuation structure 100 may be compressed by an application of a compressive force to any one or more actuation arms 110 of a plurality of actuation arms 110. Illustratively, each actuation arm 110 may be connected to one or more actuation arms 110 of a plurality of actuation arms 110 wherein an actuation of a particular actuation arm 110 may be configured to actuate every actuation arm 110 of a plurality of actuation arms 110. In one or more embodiments, one or more actuation arms 110 may be configured to actuate in pairs or groups. For example, an actuation of a first actuation arm 110 may be configured to actuate a second actuation arm 110.

Illustratively, a compression of actuation structure 100, e.g., due to an application of a force to a portion of actuation structure 100, may be configured to expand one or more extension joints 111 of a particular actuation arm 110. In one or more embodiments, an expansion of an extension joint 111 of a particular actuation arm 110 may be configured to increase a distance between a distal end and a proximal end of the particular actuation arm 110. Illustratively, an expansion of an extension joint 111 of a particular actuation arm 110 may be configured to expand an extension joint 111 of every actuation arm 110 of a plurality of actuation arms 110. In one or more embodiments, an expansion of an extension joint 111 of every actuation arm 110 of a plurality of actuation arms 110 may be configured to increase a distance between actuation structure distal end 101 and actuation structure proximal end 102.

Illustratively, a decompression of actuation structure 100, e.g., due to a reduction of a force applied to a portion of actuation structure 100, may be configured to collapse one or more extension joints 111 of a particular actuation arm 110. In one or more embodiments, a collapse of an extension joint 111 of a particular actuation arm 110 may be configured to decrease a distance between a distal end and a proximal end of the particular actuation arm 110. Illustratively, a collapse of an extension joint 111 of a particular actuation arm 110 may be configured to collapse an extension joint 111 of every actuation arm 110 of a plurality of actuation arms 110. In one or more embodiments, a collapse of an extension joint 111 of every actuation arm 110 of a plurality of actuation arms 110 may be configured to decrease a distance between actuation structure distal end 101 and actuation structure proximal end 102.

Illustratively, a compression of actuation structure 100, e.g., due to an application of a force to a portion of actuation structure 100, may be configured to expand a proximal extension hinge 112 of a particular actuation arm 110. In one or more embodiments, an expansion of a proximal extension hinge 112 of a particular actuation arm 110 may be configured to increase a distance between a distal end and a proximal end of the particular actuation arm 110. Illustratively, an expansion of a proximal extension hinge 112 of a particular actuation arm 110 may be configured to expand a proximal extension hinge 112 of every actuation arm 110 of a plurality of actuation arms 110. In one or more embodiments, an expansion of a proximal extension hinge 112 of every actuation arm 110 of a plurality of actuation arms 110 may be configured to increase a distance between actuation structure distal end 101 and actuation structure proximal end 102.

Illustratively, a decompression of actuation structure 100, e.g., due to a reduction of a force applied to a portion of actuation structure 100, may be configured to compress a proximal extension hinge 112 of a particular actuation arm 110. In one or more embodiments, a compression of a proximal extension hinge 112 of a particular actuation arm 110 may be configured to decrease a distance between a distal end and a proximal end of the particular actuation arm 110. Illustratively, a compression of a proximal extension hinge 112 of a particular actuation arm 110 may be configured to compress a proximal extension hinge 112 of every actuation arm 110 of a plurality of actuation arms 110. In one or more embodiments, a compression of a proximal extension hinge 112 of every actuation arm 110 of a plurality of actuation arms 110 may be configured to decrease a distance between actuation structure distal end 101 and actuation structure proximal end 102.

Illustratively, a compression of actuation structure 100, e.g., due to an application of a force to a portion of actuation structure 100, may be configured to expand a distal extension hinge 113 of a particular actuation arm 110. In one or more embodiments, an expansion of a distal extension hinge 113 of a particular actuation arm 110 may be configured to increase a distance between a distal end and a proximal end of the particular actuation arm 110. Illustratively, an expansion of a distal extension hinge 113 of a particular actuation arm 110 may be configured to expand a distal extension hinge 113 of every actuation arm 110 of a plurality of actuation arms 110. In one or more embodiments, an expansion of a distal extension hinge 113 of every actuation arm 110 of a plurality of actuation arms 110 may be configured to increase a distance between actuation structure distal end 101 and actuation structure proximal end 102.

Illustratively, a decompression of actuation structure 100, e.g., due to a reduction of a force applied to a portion of actuation structure 100, may be configured to compress a distal extension hinge 113 of a particular actuation arm 110. In one or more embodiments, a compression of a distal extension hinge 113 of a particular actuation arm 110 may be configured to decrease a distance between a distal end and a proximal end of the particular actuation arm 110. Illustratively, a compression of a distal extension hinge 113 of a particular actuation arm 110 may be configured to compress a distal extension hinge 113 of every actuation arm 110 of a plurality of actuation arms 110. In one or more embodiments, a compression of a distal extension hinge 113 of every actuation arm 110 of a plurality of actuation arms 110 may be configured to decrease a distance between actuation structure distal end 101 and actuation structure proximal end 102.

Illustratively, a compression of actuation structure 100, e.g., due to an application of a force to a portion of actuation structure 100, may be configured to expand an extension joint 111, a proximal extension hinge 112, and a distal extension hinge 113 of a particular actuation arm 110. In one or more embodiments, an expansion of an extension joint 111, a proximal extension hinge 112, and a distal extension hinge 113 of a particular actuation arm 110 may be configured to increase a distance between a distal end and a proximal end of the particular actuation arm 110. Illustratively, an expansion of an extension joint 111, a proximal extension hinge 112, and a distal extension hinge 113 of a particular actuation arm 110 may be configured to expand an extension joint 111, a proximal extension hinge 112, and a distal extension hinge 113 of every actuation arm 110 of a plurality of actuation arms 110. In one or more embodiments, an expansion of an extension joint 111, a proximal extension hinge 112, and a distal extension hinge 113 of every actuation arm 110 of a plurality of actuation arms 110 may be configured to increase a distance between actuation structure distal end 101 and actuation structure proximal end 102.

Illustratively, a decompression of actuation structure 100, e.g., due to a reduction of a force applied to a portion of actuation structure 100, may be configured to compress an extension joint 111, a proximal extension hinge 112, and a distal extension hinge 113 of a particular actuation arm 110. In one or more embodiments, a compression of an extension joint 111, a proximal extension hinge 112, and a distal extension hinge 113 of a particular actuation arm 110 may be configured to decrease a distance between a distal end and a proximal end of the particular actuation arm 110. Illustratively, a compression of an extension joint 111, a proximal extension hinge 112, and a distal extension hinge 113 of a particular actuation arm 110 may be configured to compress an extension joint 111, a proximal extension hinge 112, and a distal extension hinge 113 of every actuation arm 110 of a plurality of actuation arms 110. In one or more embodiments, a compression of an extension joint 111, a proximal extension hinge 112, and a distal extension hinge 113 of every actuation arm 110 of a plurality of actuation arms 110 may be configured to decrease a distance between actuation structure distal end 101 and actuation structure proximal end 102.

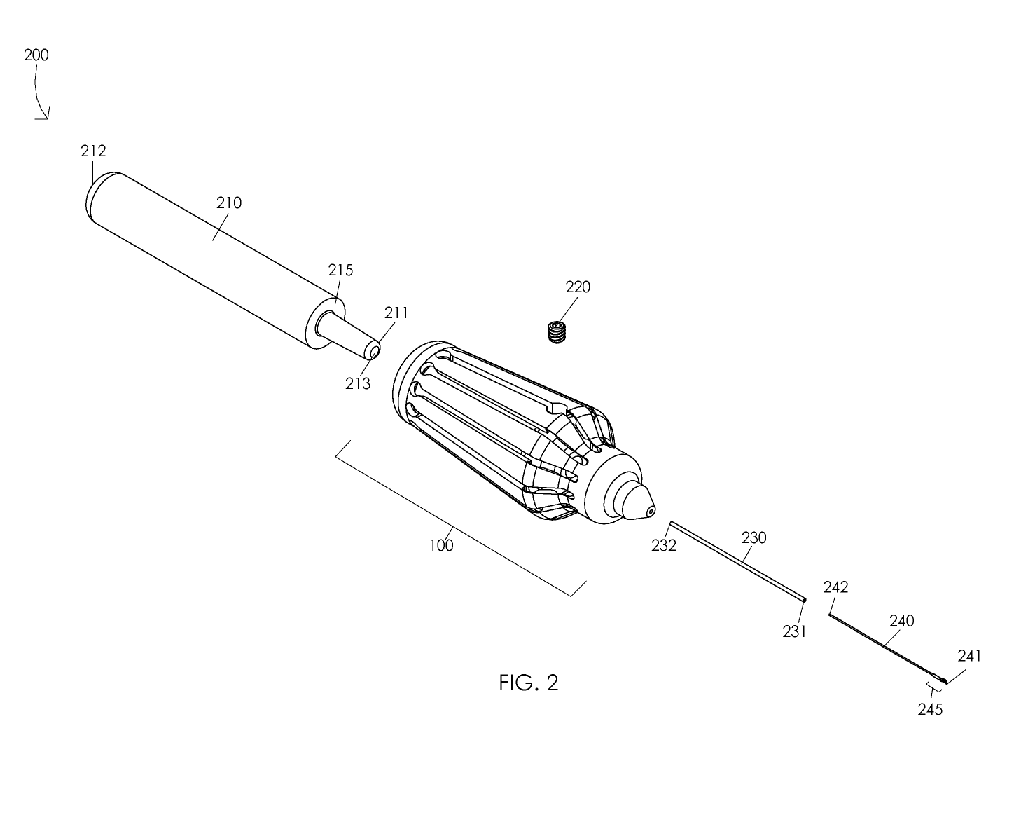

FIG. 2 is a schematic diagram illustrating an exploded view of a surgical instrument assembly 200. Illustratively, a surgical instrument assembly 200 may comprise an actuation structure 100, a handle base 210 having a handle base distal end 211 and a handle base proximal end 212, a fixation mechanism 220, an actuation sleeve 230 having an actuation sleeve distal end 231 and an actuation sleeve proximal end 232, and a surgical blank 240 having a surgical blank distal end 241 and a surgical blank proximal end 242. In one or more embodiments, surgical blank 240 may comprise a surgical tool 245, e.g., a forceps, a scissors, etc. Illustratively, handle base 210 may comprise a handle base inner bore 213 and an actuation structure proximal interface 215.

In one or more embodiments, handle base 210, actuation sleeve 230, and surgical blank 240 may be manufactured from a material suitable for sterilization by a medical autoclave. Illustratively, handle base 210, actuation sleeve 230, and surgical blank 240 may be manufactured from a material configured to withstand exposure to temperatures, pressures, and ambient conditions present in a medical autoclave without degradation. For example, handle base 210, actuation sleeve 230, and surgical blank 240 may be configured to function normally after exposure in a temperature 250° F. for 15 minutes at an atmospheric pressure of 15 psi. In one or more embodiments, handle base 210, actuation sleeve 230, and surgical blank 240 may be configured to be used in a surgical procedure and then sterilized by a medical autoclave at least three times. Illustratively, handle base 210, actuation sleeve 230, and surgical blank 240 may be configured to be used in a surgical procedure and then sterilized by a medical autoclave more than three times.

In one or more embodiments, a portion of handle base 210 may be disposed within actuation structure, e.g., handle base distal end 211 may be disposed within inner bore 130. Illustratively, handle base 210 may be disposed within inner bore 130 wherein actuation structure proximal interface 215 abuts actuation structure proximal end 102. In one or more embodiments, handle base 210 may be fixed to actuation structure proximal end 102, e.g., actuation structure proximal interface 215 may be fixed to actuation structure proximal end 102. Illustratively, a portion of handle base 210 may be fixed to a portion of actuation structure 100, e.g., by an adhesive or any suitable fixation means. Illustratively, a portion of handle base 210 may be fixed within a portion of actuation structure 100, e.g., a portion of handle base 210 may be fixed within inner bore 130. In one or more embodiments, a portion of handle base 210 may be fixed within a portion of actuation structure 100, e.g., by an adhesive or any suitable fixation means. For example, a portion of handle base 210 may be fixed within a portion of actuation structure 100 by a press fit, a screw threading, a weld, etc.

Illustratively, a portion of actuation sleeve 230 may be fixed to a portion of actuation structure 100, e.g., actuation sleeve proximal end 232 may be fixed to actuation structure distal end 101. In one or more embodiments, a portion of actuation sleeve 230 may be fixed to a portion of actuation structure 100, e.g., by an adhesive or any suitable fixation means. Illustratively, a portion of actuation sleeve 230 may be disposed within a portion of actuation structure 100, e.g., actuation sleeve proximal end 232 may be disposed within actuation sleeve housing 150. In one or more embodiments, a portion of actuation sleeve 230 may be fixed within a portion of actuation structure 100, e.g., by an adhesive or any suitable fixation means. For example, a portion of actuation sleeve 230 may be fixed within a portion of actuation structure 100 by a press fit, a screw threading, a weld, etc.

Illustratively, surgical blank 240 may be disposed within actuation sleeve 230, actuation sleeve housing 150, inner bore 130, and fixation mechanism housing 115. In one or more embodiments, a portion of surgical blank 240 may be fixed in a position relative to actuation structure 100. Illustratively, fixation mechanism 220 may be configured to fix surgical blank 240 in a position relative to actuation structure 100, e.g., fixation mechanism 220 may be disposed within fixation mechanism housing 115. In one or more embodiments, fixation mechanism 220 may be configured to fix a portion of surgical blank 240 within fixation mechanism housing 115. Illustratively, fixation mechanism 220 may comprise a setscrew configured to firmly fix a portion of surgical blank 240 within fixation mechanism housing 115. In one or more embodiments, surgical blank 240 may be fixed within fixation mechanism housing 115, e.g., by an adhesive or any suitable fixation means.

Illustratively, a compression of actuation structure 100 may be configured to extend actuation structure distal end 101 relative to actuation structure proximal end 102. In one or more embodiments, an extension of actuation structure distal end 101 relative to actuation structure proximal end 102 may be configured to extend actuation sleeve housing 150 relative to actuation structure proximal end 102. Illustratively, an extension of actuation sleeve housing 150 relative to actuation structure proximal end 102 may be configured to extend actuation sleeve 230 relative to actuation structure proximal end 102. In one or more embodiments, an extension of actuation sleeve 230 relative to actuation structure proximal end 102 may be configured to extend actuation sleeve 230 relative to surgical blank 240. Illustratively, an extension of actuation sleeve 230 relative to surgical blank 240 may be configured to extend actuation sleeve distal end 231 relative to surgical tool 245. In one or more embodiments, an extension of actuation sleeve distal end 231 relative to surgical tool 245 may be configured to gradually close surgical tool 245, e.g., a compression of actuation structure 100 may be configured to gradually close surgical tool 245.

Illustratively, a decompression compression of actuation structure 100 may be configured to retract actuation structure distal end 101 relative to actuation structure proximal end 102. In one or more embodiments, a retraction of actuation structure distal end 101 relative to actuation structure proximal end 102 may be configured to retract actuation sleeve housing 150 relative to actuation structure proximal end 102. Illustratively, a retraction of actuation sleeve housing 150 relative to actuation structure proximal end 102 may be configured to retract actuation sleeve 230 relative to actuation structure proximal end 102. In one or more embodiments, a retraction of actuation sleeve 230 relative to actuation structure proximal end 102 may be configured to retract actuation sleeve 230 relative to surgical blank 240. Illustratively, a retraction of actuation sleeve 230 relative to surgical blank 240 may be configured to retract actuation sleeve distal end 231 relative to surgical tool 245. In one or more embodiments, a retraction of actuation sleeve distal end 231 relative to surgical tool 245 may be configured to gradually open surgical tool 245, e.g., a decompression of actuation structure 100 may be configured to gradually open surgical tool 245.

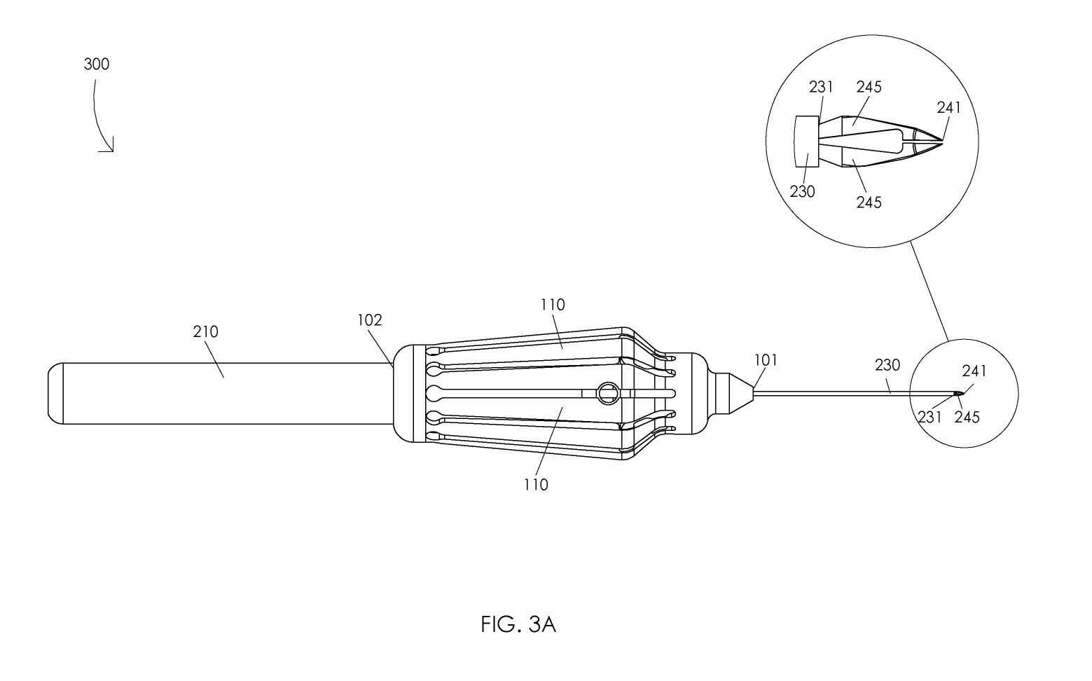

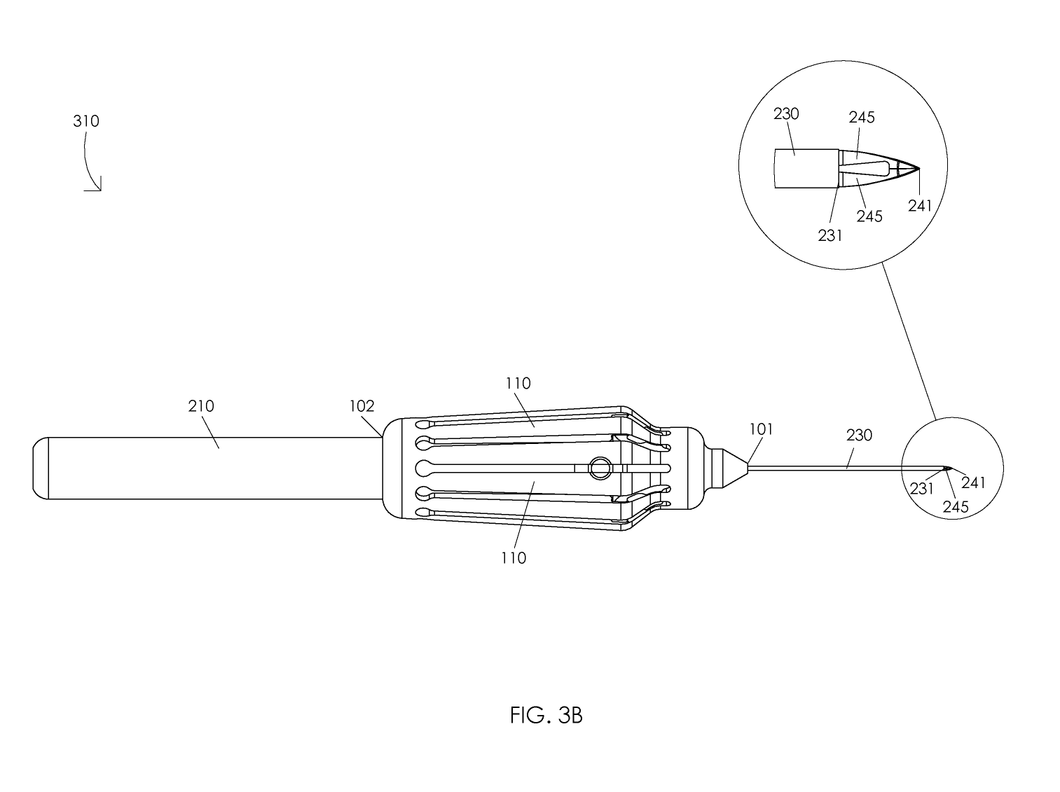

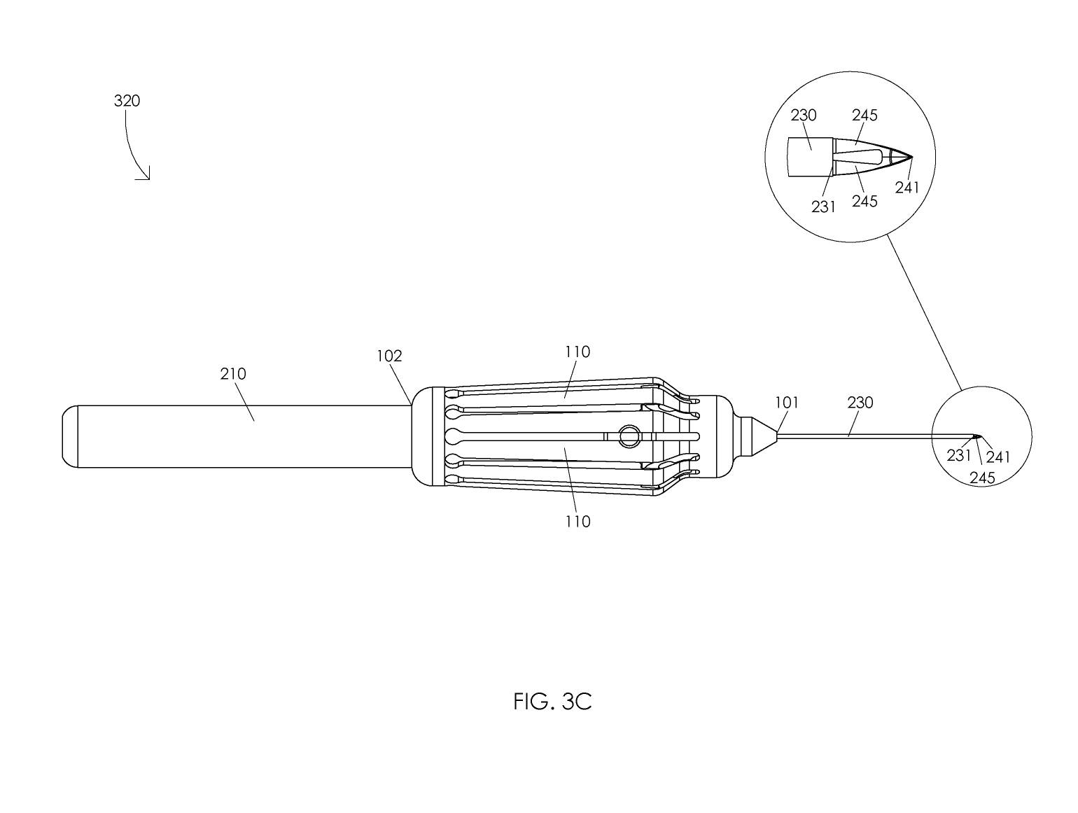

FIGS. 3A, 3B, and 3C are schematic diagrams illustrating a gradual closing of a surgical tool 245. FIG. 3A illustrates an open surgical tool 300. Illustratively, surgical tool 245 may comprise an open surgical tool 300, e.g., when actuation structure 100 is fully decompressed. In one or more embodiments, surgical tool 245 may comprise an open surgical tool 300, e.g., when actuation structure distal end 101 is fully retracted relative to actuation structure proximal end 102. Illustratively, surgical tool 245 may comprise an open surgical tool 300, e.g., when actuation sleeve 230 is fully retracted relative to surgical blank 240. In one or more embodiments, surgical tool 245 may comprise an open surgical tool 300, e.g., when actuation sleeve 230 is fully retracted relative to surgical tool 245. Illustratively, surgical tool 245 may comprise an open surgical tool 300, e.g., when no force is applied to actuation structure 100.

FIG. 3B illustrates a partially closed surgical tool 310. Illustratively, a compression of actuation structure 100 may be configured to gradually close surgical tool 245 from an open surgical tool 300 to a partially closed surgical tool 310. In one or more embodiments, a surgeon may compress actuation structure 100, e.g., by applying a force to a portion of actuation structure 100. Illustratively, a compression of actuation structure 100 may be configured to gradually extend actuation structure distal end 101 relative to actuation structure proximal end 102. In one or more embodiments, an extension of actuation structure distal end 101 relative to actuation structure proximal end 102 may be configured to extend actuation sleeve 230 relative to surgical blank 240. Illustratively, an extension of actuation sleeve 230 relative to surgical blank 240 may be configured to extend a portion of actuation sleeve 230 over a portion of surgical tool 245. In one or more embodiments, an extension of a portion of actuation sleeve 230 over a portion of surgical tool 245 may be configured to gradually close surgical tool 245. Illustratively, an extension of a portion of actuation sleeve 230 over a portion of surgical tool 245 may be configured to gradually close surgical tool 245, e.g., from an open surgical tool 300 to a partially closed surgical tool 310.

FIG. 3C illustrates a fully closed surgical tool 320. Illustratively, a compression of actuation structure 100 may be configured to gradually close surgical tool 245 from a partially closed surgical tool 310 to a fully closed surgical tool 320. In one or more embodiments, a surgeon may compress actuation structure 100, e.g., by applying a force to a portion of actuation structure 100. Illustratively, a compression of actuation structure 100 may be configured to gradually extend actuation structure distal end 101 relative to actuation structure proximal end 102. In one or more embodiments, an extension of actuation structure distal end 101 relative to actuation structure proximal end 102 may be configured to extend actuation sleeve 230 relative to surgical blank 240. Illustratively, an extension of actuation sleeve 230 relative to surgical blank 240 may be configured to extend a portion of actuation sleeve 230 over a portion of surgical tool 245. In one or more embodiments, an extension of a portion of actuation sleeve 230 over a portion of surgical tool 245 may be configured to gradually close surgical tool 245. Illustratively, an extension of a portion of actuation sleeve 230 over a portion of surgical tool 245 may be configured to gradually close surgical tool 245, e.g., from a partially closed surgical tool 310 to a fully closed surgical tool.

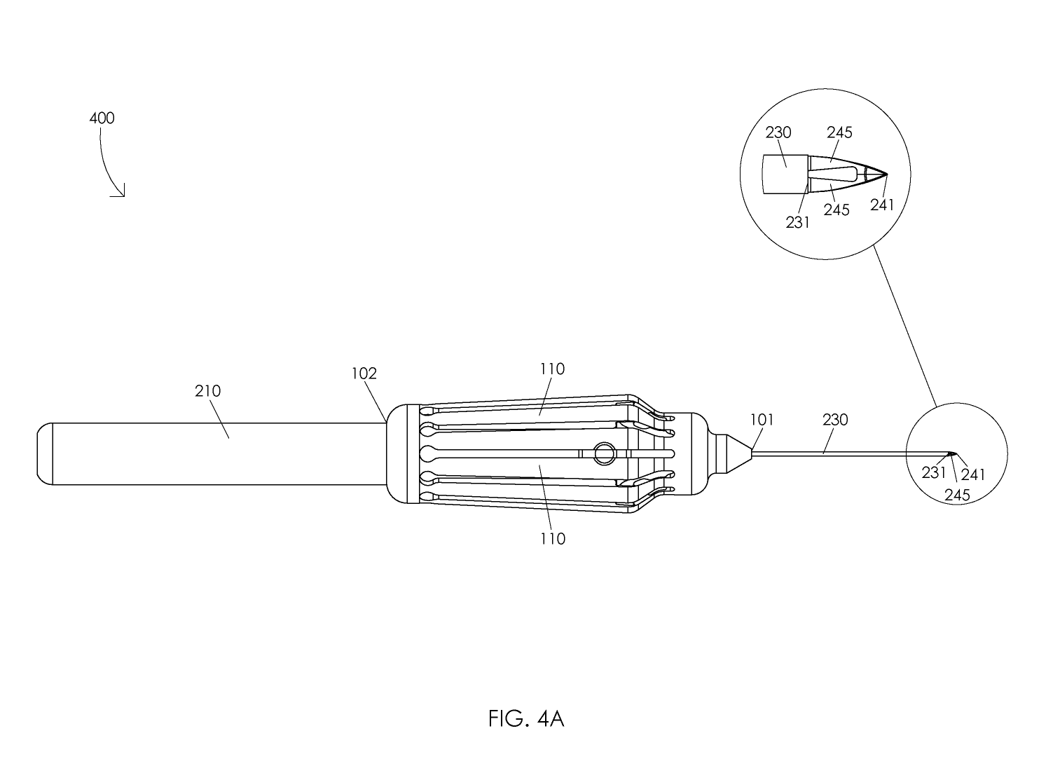

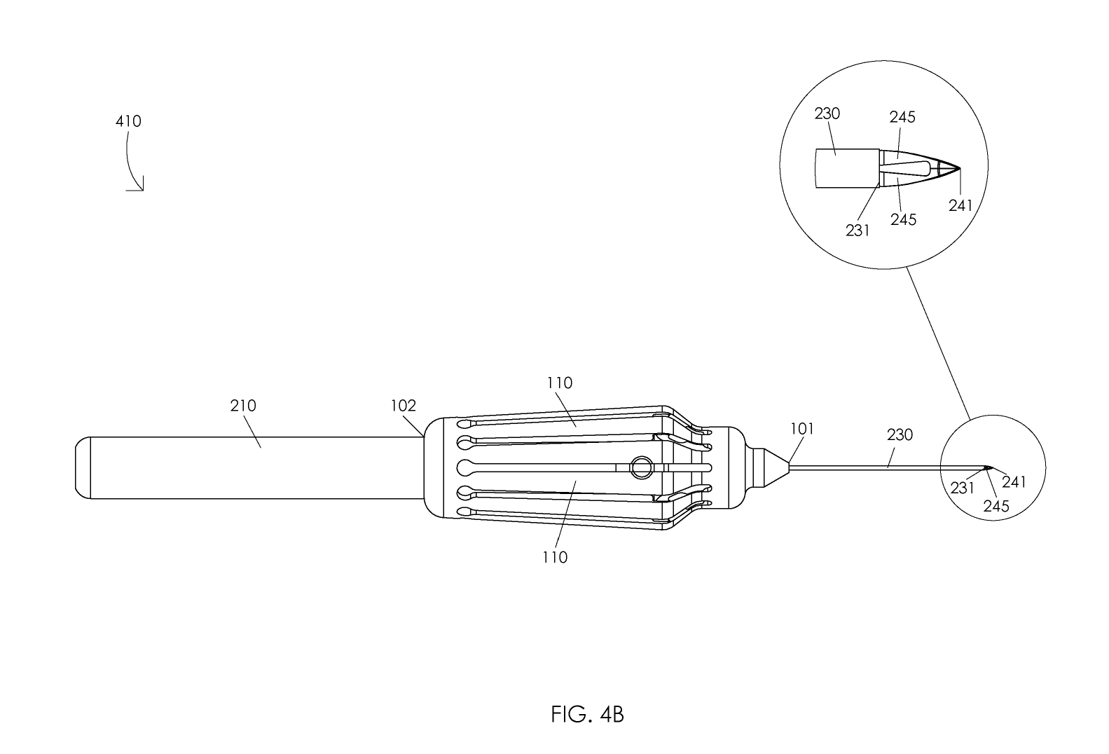

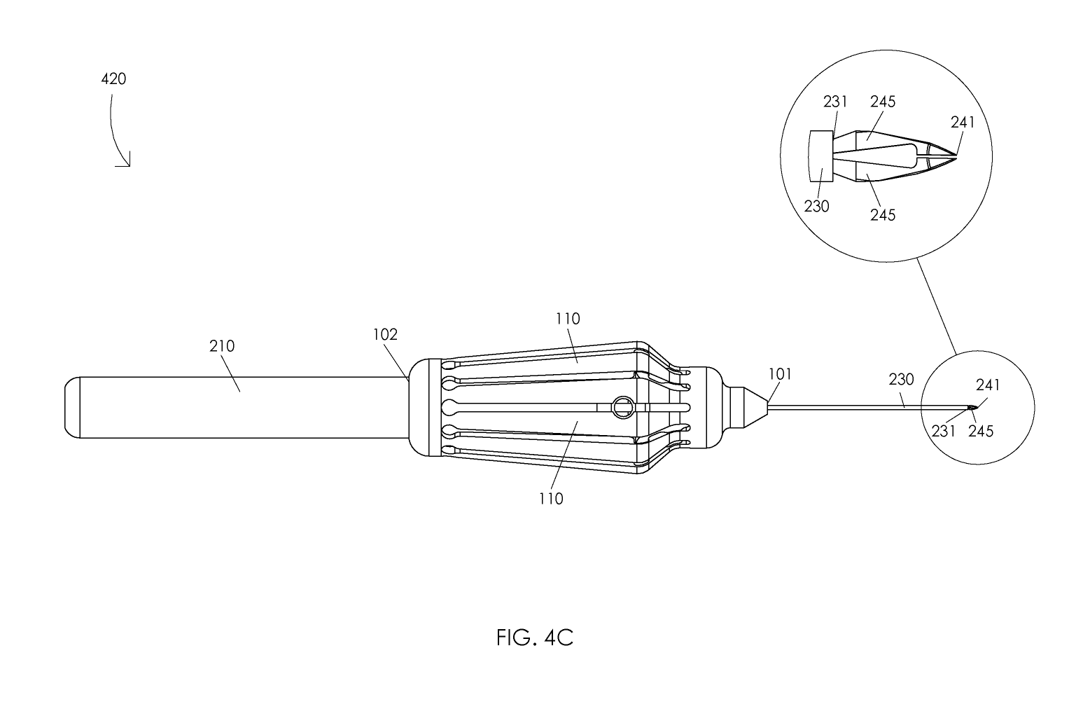

FIGS. 4A, 4B, and 4C are schematic diagrams illustrating a gradual opening of a surgical tool 245. FIG. 4A illustrates a closed surgical tool 400. Illustratively, surgical tool 245 may comprise a closed surgical tool 400, e.g., when actuation structure 100 is fully compressed. In one or more embodiments, surgical tool 245 may comprise a closed surgical tool 400, e.g., when actuation structure distal end 101 is fully extended relative to actuation structure proximal end 102. Illustratively, surgical tool 245 may comprise a closed surgical tool 400, e.g., when actuation sleeve 230 is fully extended relative to surgical blank 240. In one or more embodiments, surgical tool 245 may comprise a closed surgical tool 400, e.g., when actuation sleeve 230 is fully extended relative to surgical tool 245. Illustratively, surgical tool 245 may comprise a closed surgical tool 400, e.g., when a compression force is applied to actuation structure 100.

FIG. 4B illustrates a partially open surgical tool 410. Illustratively, a decompression of actuation structure 100 may be configured to gradually open surgical tool 245 from a closed surgical tool 400 to a partially open surgical tool 410. In one or more embodiments, a surgeon may decompress actuation structure 100, e.g., by reducing a force applied to a portion of actuation structure 100. Illustratively, a decompression of actuation structure 100 may be configured to gradually retract actuation structure distal end 101 relative to actuation structure proximal end 102. In one or more embodiments, a retraction of actuation structure distal end 101 relative to actuation structure proximal end 102 may be configured to retract actuation sleeve 230 relative to surgical blank 240. Illustratively, a retraction of actuation sleeve 230 relative to surgical blank 240 may be configured to retract a portion of actuation sleeve 230 away from a portion of surgical tool 245. In one or more embodiments, a retraction of a portion of actuation sleeve 230 away from a portion of surgical tool 245 may be configured to gradually open surgical tool 245. Illustratively, a retraction of a portion of actuation sleeve 230 away from a portion of surgical tool 245 may be configured to gradually open surgical tool 245, e.g., from a closed surgical tool 400 to a partially open surgical tool 410.

FIG. 4C illustrates a fully open surgical tool 420. Illustratively, a decompression of actuation structure 100 may be configured to gradually open surgical tool 245 from a partially open surgical tool 410 to a fully open surgical tool 420. In one or more embodiments, a surgeon may decompress actuation structure 100, e.g., by reducing a force applied to a portion of actuation structure 100. Illustratively, a decompression of actuation structure 100 may be configured to gradually retract actuation structure distal end 101 relative to actuation structure proximal end 102. In one or more embodiments, a retraction of actuation structure distal end 101 relative to actuation structure proximal end 102 may be configured to retract actuation sleeve 230 relative to surgical blank 240. Illustratively, a retraction of actuation sleeve 230 relative to surgical blank 240 may be configured to retract a portion of actuation sleeve 230 away from a portion of surgical tool 245. In one or more embodiments, a retraction of a portion of actuation sleeve 230 away from a portion of surgical tool 245 may be configured to gradually open surgical tool 245. Illustratively, a retraction of a portion of actuation sleeve 230 away from a portion of surgical tool 245 may be configured to gradually open surgical tool 245, e.g., from a partially open surgical tool 410 to a fully open surgical tool 420.

In one or more embodiments, one or more properties of a surgical instrument handle may be adjusted to attain one or more desired surgical instrument handle features. Illustratively, handle base 210 may be removed from actuation structure 100, e.g., handle base 210 may be removed from actuation structure 100 during a surgical procedure. In one or more embodiments, a surgeon may utilize handle base 210 to steady surgical tool 245, e.g., during a portion of a surgical procedure. Illustratively, a surgeon may remove handle base 210 and manipulate actuation structure 100 to open or close surgical tool 245, e.g., during a portion of a surgical procedure. In one or more embodiments, handle base 210 may be added to actuation structure 100, e.g., handle base 210 may be added to actuation structure 100 during a surgical procedure. Illustratively, surgical tool 245 may comprise a surgical forceps. In one or more embodiments, surgical tool 245 may comprise a surgical scissors.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a surgical instrument, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
an actuation structure having an actuation structure distal end and an actuation structure proximal end, the actuation structure having a density in a range of 0.02 to 0.06 pounds per cubic inch;
a plurality of actuation arms of the actuation structure, each actuation arm of the plurality of actuation arms having an extension joint, a distal extension hinge, and a proximal extension hinge;
a handle base having a handle base distal end and a handle base proximal end; and
an actuation sleeve having an actuation sleeve distal end and an actuation sleeve proximal end, the actuation sleeve disposed in an actuation sleeve housing of the actuation structure.

2. The instrument of claim 1 wherein the actuation structure has a mass in a range of 0.005 to 0.025 pounds.

3. The instrument of claim 1 wherein the actuation structure has a volume in a range of 0.2 to 0.5 cubic inches.

4. The instrument of claim 1 wherein the actuation structure has a surface area in a range of 7.5 to 13.0 square inches.

5. The instrument of claim 1 further comprising:
a surgical blank having a surgical blank distal end and a surgical blank proximal end, the surgical blank disposed in the actuation structure and the actuation sleeve; and
a surgical tool of the surgical blank.

6. The instrument of claim 5 wherein a compression of the actuation structure is configured to gradually close the surgical tool.

7. The instrument of claim 6 wherein the compression of the actuation structure is configured to extend the actuation structure distal end relative to the actuation structure proximal end.

8. The instrument of claim 7 wherein the compression of the actuation structure is configured to extend the actuation structure distal end in a range of 0.02 to 0.05 inches from the actuation structure proximal end.

9. The instrument of claim 7 wherein the compression of the actuation structure is configured to expand the extension joint of each actuation arm of the plurality of actuation arms.

10. The instrument of claim 7 wherein the compression of the actuation structure is configured to extend the actuation sleeve relative to the surgical blank.

11. The instrument of claim 5 wherein a decompression of the actuation structure is configured to gradually open the surgical tool.

12. The instrument of claim 11 wherein the decompression of the actuation structure is configured to retract the actuation structure distal end relative to the actuation structure proximal end.

13. The instrument of claim 12 wherein the decompression of the actuation structure is configured to retract the actuation structure distal end in a range of 0.02 to 0.05 inches from the actuation structure proximal end.

14. The instrument of claim 12 wherein the decompression of the actuation structure is configured to collapse the extension joint of each actuation arm of the plurality of actuation arms.

15. The instrument of claim 12 wherein the decompression of the actuation structure is configured to retract the actuation sleeve relative to the surgical blank.

16. The instrument of claim 5 wherein the surgical tool comprises a surgical forceps.

17. The instrument of claim 5 wherein the surgical tool comprises a surgical scissors.

18. A method comprising:
applying a compressive force in a range of 0.2 to 1.0 pounds to a portion of an actuation structure;
compressing the actuation structure;
extending a distal end of the actuation structure in a range of 0.02 to 0.05 inches relative to a proximal end of the actuation structure;
extending an actuation sleeve relative to a surgical blank; and
closing a surgical tool.

19. The method of claim 18 further comprising:
removing the compressive force from the portion of the actuation structure;
decompressing the actuation structure;
retracting the distal end of the actuation structure relative to the proximal end of the actuation structure;
retracting the actuation sleeve relative to the surgical blank; and
opening a surgical tool.

20. The method of claim 19 wherein the actuation structure has a density in a range of 0.02 to 0.06 pounds per cubic inch.
